(19) United States
(12) Patent Application Publication
(10) Pub. No.: US 2026/0300887 A1
(43) Pub. Date: Oct. 1, 2026

(54) ARTIFICIAL INTELLIGENCE SYSTEMS AND METHODS FOR TALENT DEVELOPMENT AND TALENT ACQUISITION

(71) Applicant: IGNIS AI, LLC, Surfside, FL (US)

(72) Inventors: Yigal Rosen, Brookline, MA (US); Avraham Kadar, Surfside, FL (US); Ilia P. Rushkin, Portland, ME (US)

(73) Assignee: IGNIS AI, LLC, Surfside, FL (US)

(21) Appl. No.: 19/575,286

(22) Filed: Mar. 23, 2026

Related U.S. Application Data
(60) Provisional application No. 63/777,453, filed on Mar. 25, 2025.

(51) Int. Cl. G06Q 10/0639; G06Q 10/0631

(52) U.S. Cl. CPC G06Q 10/06393; G06Q 10/063112

(57) ABSTRACT

A system uses artificial intelligence to generate comprehensive skill profiles for individuals, to aid talent development and evaluate compatibility with job positions. In some embodiments, individuals create profiles through a graphical user interface containing biographical information, experience, education, and other materials. AI models may extract explicit and inferred skills and categorize them with a standardized skills ontology. In selected embodiments, the system calculates proficiencies for each skill, assigns confidence levels to skills based on supporting evidence, and verifies skills through automated assessments. In some embodiments, employers create job profiles that include titles, descriptions, and requirements, from which AI models infer additional associated skills. In some embodiments, a matching engine compares and individual's skills with job requirements to calculate suitability for the position, enabling improved talent development and acquisition decisions.

### CROSS-REFERENCE TO RELATED APPLICATIONS

This application claims the benefit of U.S. provisional application Ser. No. 63/777,452, filed Mar. 25, 2025, entitled Artificial Intelligence Methods for Automated Optimization of Talent Acquisition and Talent Management Systems, the entirety of which is incorporated by reference herein.

### BACKGROUND

Accurate evaluation of an individual's skills is important for supporting professional development. Individuals may benefit from understanding their own capabilities in order to improve existing skills, acquire new competencies, and pursue appropriate career opportunities. Employers similarly benefit from reliable skill assessments when developing employees or selecting candidates for open job positions that require particular competencies.

Individuals commonly create personal profiles that include descriptions of education, work experience, accomplishments, and claimed skills. Such profiles may be provided in resumes, professional networking platforms, application forms, or other structured or unstructured documents. However, these profiles are often incomplete or inconsistent.

Individuals may manually list skills using free-form text or from predefined lists, but such listings frequently fail to capture the full range of an individual's capabilities. For example, profiles may omit cross-functional competencies and soft skills that are not traditionally listed but nonetheless contribute to job performance. In addition, skills that are related, complementary, or prerequisite to other skills may not be identified.

Another challenge arises from the self-reported nature of many skill descriptions. Individuals may unintentionally misjudge their own level of proficiency or present information in a manner that does not accurately reflect their abilities. As a result, the listed skills may lack verification or objective measurement. Without reliable assessment mechanisms, both individuals and employers may have limited visibility into the true level of proficiency associated with a given skill.

Incomplete or inaccurate skill information may also prevent individuals from recognizing gaps in their capabilities relative to current or desired roles. When individuals lack insight into these gaps, opportunities for targeted development may be missed, potentially slowing professional growth. Employers likewise may face difficulties when attempting to develop employees or evaluate candidates, particularly when the available information does not clearly indicate the presence or level of relevant skills.

Employers may also experience challenges in defining the skills required for a particular job position. Job descriptions and internal competency frameworks may identify certain technical capabilities but may overlook other relevant competencies, such as cross-functional or soft skills, that contribute to successful performance in the role. In addition, relationships between skills, including prerequisite skills or commonly associated competencies, may not be explicitly represented. As a result, evaluating whether an individual is well suited for a particular position may be difficult.

Accordingly, there exists a need for improved systems and methods capable of identifying, inferring, assessing, and verifying a comprehensive set of skills associated with an individual, including both technical and soft skills. There is further a need for techniques that can estimate levels of proficiency for such skills, identify relationships among skills, and compare an individual's capabilities with the skills required for a particular job position. Systems addressing these needs would support more accurate skill assessment, facilitate talent development, and improve the alignment between individuals and job opportunities.

### SUMMARY OF ILLUSTRATIVE EMBODIMENTS

To overcome the above-noted limitations, the inventors recognized a need for an artificial intelligence automated method of generating complete lists of skills, in addition to other factors, such as assessments of experience, training, and education, as possessed by an individual or desired by an employer. In some embodiments, with this information, a proficiency calculation of an individual's skills is provided in real-time and a job fit score between the individual and a given job position. Further, the inventors have developed a method of quantifying and verifying an individual's skills so the individual and/or an employer can make informed decisions in the next step of the talent development or acquisition process. Hereinafter, the term “individual” may refer to an independent user, an employee, a candidate, a student, or any other person.

In some embodiments, individuals interact with the system through a graphical user interface to create profiles that include biographical details, work experience, education, and uploaded digital materials. The system assists individuals in completing their profiles by prompting for additional information when needed. AI models extract both explicit and inferred skills from the individual's data, categorizing them into Generic Skills and Power Skills. Generic Skills are analogous to core technical competencies or hard skills. Power Skills are analogous to cross-functional competencies or soft skills, which include both intrapersonal skills and interpersonal skills. These skills are validated and stored in the individual's profile using a standardized skills ontology.

For individuals, in some embodiments, the system continuously updates inferred skills by analyzing related job titles, skills clusters, and updated individual information. Confidence levels are assigned to each inferred skill based on supporting data, such as years of experience, certifications, or prior job descriptions. The system prompts individuals for clarification if a skill has a low confidence level, allowing the system to refine the confidence level over time.

In some embodiments, an AI-powered chatbot conducts skill verification by prompting individuals with assessment questions. A dynamic question bank is used to generate varied and structured questions, such as multiple-choice, ranking, or open-ended questions. Responses affect the properties of the individual's skills, thereby adjusting confidence levels and proficiency distributions. In some embodiments, once assessments are complete, the system generates a verified JobSkillsPrint, a master record of an individual's confirmed skills and competencies.

In some embodiments, a Career Pathway Engine recommends an individual improve proficiency levels of skills or attain new skills, by identifying skills to further their career in their current position or alternative positions. The Career Pathway Engine helps them determine areas of weakness that may be bolstered with additional skills, as well as areas of strength that may demonstrate their suitability, with a few select skill improvements, for previously unconsidered positions. In some embodiments, the system provides employers and individuals with visibility into individuals' Generic Skills and Power Skills, and provides a talent development program for setting goals and reaching high potentials in skill acquisition.

In some embodiments, an automated inference and assessment system efficiently matches individuals with job positions by processing data from both individuals and employers. It may include several AI-driven processing engines that perform tasks such as building individual profiles, defining job position requirements, and evaluating the fitness of individuals for specific roles. Key components present in many embodiments include user interfaces for individuals and employers, AI-powered chatbots, skills inference models, confidence level assessments, and recommendation engines, all working together to streamline talent acquisition.

Similarly, in some embodiments, employers use an interface to create job position profiles, specifying job titles, descriptions, required skills, and other relevant details. The system aids in profile completion by prompting employers for additional input. AI models infer skills from job descriptions and related job postings, creating a comprehensive list of required skills. Standardized skills ontologies ensure consistency in skill classification across various job listings.

To improve the comprehensiveness and accuracy of inferred skills, in some embodiments, the system compares job postings with similar positions, identifying overlooked skills that are commonly associated with specific roles. AI models generate lists of additional inferred skills, ensuring that both Generic Skills and Power Skills are fully accounted for in the job position profile.

In some embodiments, the system also automatically evaluates how well an individual fits a job position by comparing verified skills against job requirements. A JobSuccessFit Engine calculates a JobSuccessFit score, ranking individuals based on their suitability for specific roles. Employers can view top-matching individuals, while individuals can see which roles best align with their skill sets.

In some embodiments, this AI-driven talent acquisition platform continuously learns from employer and individual interactions. By analyzing vast amounts of data, it refines its skills inference, individual assessment, and job matching models over time. The platform dynamically updates both individual profiles and job profiles to reflect new skills, experiences, and changing industry demands, ensuring an ever-improving hiring process that benefits both employers and individuals.

The foregoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a block diagram of an example platform for automatically inferring and assessing individuals for job positions;

FIG. 2 illustrates a flow chart of example methods for building an individual profile with individual-provided materials;

FIG. 3 illustrates a flow chart of example methods for building a set of required skills for a job position;

FIG. 4 illustrates a flow chart of example methods for building an individual profile with inferred skills from related job titles and associated skills;

FIG. 5 illustrates a flow chart of example methods for building an individual's set of unverified skills from feedback prompts;

FIG. 6 illustrates a flow chart of example methods for assessing and verifying an individual's set of skills through question prompts;

FIG. 7 is a data flow diagram of an example process for assessing and verifying an individual's set of skills through question prompts;

FIG. 8 is a data flow diagram of an example process for assessing skill proficiency;

FIG. 9 is a data flow diagram of an example process for updating skill proficiency distributions;

FIG. 10 shows seven example skill proficiency distributions;

FIG. 11 illustrates a flow chart of example methods for calculating an individual's successful fit with a job position;

FIG. 12 shows a conceptual diagram of a pyramid, demonstrating one embodiment for determining an individual's fit to a job position;

FIG. 13 is a high-level data flow diagram of an example process calculating an individual's successful fit with a job position;

FIG. 14 illustrates elements of an example career pathway user interface; and

FIG. 15 illustrates elements of an example performance review user interface.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

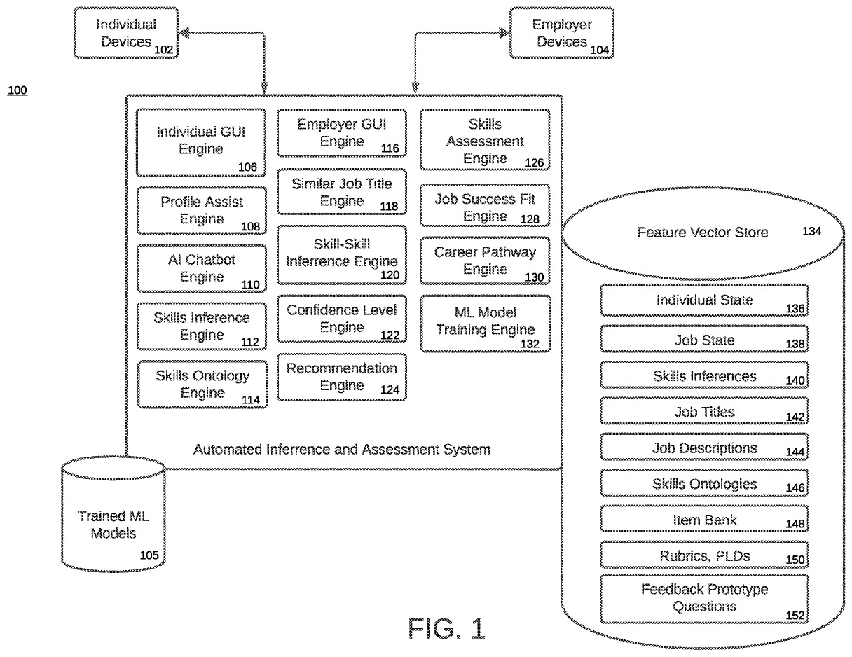

FIG. 1 is a block diagram of an example platform 100 for automatically inferring and assessing individuals skills, for talent development and fitness for job positions. An individual may refer to an independent user, an employee, a candidate, a student, or any other person. In some embodiments, an automated inference and assessment system interacts with Individual Devices 102 and employer devices 104. The system maintains several processing engines, Trained Machine Learning (ML) Models 105, which perform various tasks related to building individual profiles, building job position requirements, assessing an individual's skills, recommending development of skills, and the fitness between individuals and job positions. Processing engines include the Individual Graphical User Interface (GUI) Engine 106, the Profile Assist Engine 108, the AI Chatbot Engine 110, the Skills Inference Engine 112, the Skills Ontology Engine 114, the Employer GUI Engine 116, the Similar Job Title Engine 118, the Skill-Skill Inference Engine 120, the Confidence Level Engine 122, the Recommendation Engine 124, the Skills Assessment Engine 126, the Job Success Fit Engine 128, the Career Pathway Engine 130, and the ML Model Training Engine 132.

The Individual GUI Engine 106 provides a user interface for an individual to create a profile and interact with the system. The Profile Assist Engine 108 aids individuals and employers in creating profiles by prompting for more information when appropriate. The AI Chatbot Engine 110 serves as a prompt engine for the Profile Assist Engine 108 and executes processes for inferring skills, assigning properties to skills, and verifying skills, among others. The Skills Inference Engine 112 is an AI model which extracts skills from various inputs provided by individuals, and develops Skill Inferences 140 over time through machine learning. In some embodiments, the Skills Inference Engine 112 is trained with data including, but not limited to, individual profiles (biographical information, work history, education), uploaded digital materials (resumes, cover letters, project descriptions, audio, video, code, graphics), job titles, and job descriptions. It also learns from accumulated skill inferences over time as individuals interact with the system.

The Skills Ontology Engine 114 creates a standardized ontology of skills, providing connections between related skills and prerequisite skills. The Employer GUI Engine 116 provides a user interface for an employer to create a job post and interact with the system. The Similar Job Title Engine 118 is an AI model which finds job positions in a family of jobs with similar skill sets to a newly added job position. In some embodiments, the Similar Job Title Engine 118 is trained with data including, but not limited to, job titles, job descriptions, and associated skill sets across a broad range of job positions and industries.

The Skill-Skill Inference Engine 120 is an AI model which generates an additional list of inferred skills from a first list of skills. Examples could be prerequisite skills, cross-functional competency skills, or other associated skills that generally would cluster around the first list of inferred skills. In some embodiments, the Skill-Skill Inference Engine 120 is trained with data including skills ontologies, skill-to-skill relationships and hierarchies, job postings, and historical patterns of skills that commonly co-occur or cluster together.

The Confidence Level Engine 122 is an AI model which assigns a level of confidence as a property of a skill for a given individual, based on information provided by the individual and confidence levels for other individuals with similar experience. In some embodiments, the Confidence Level Engine 122 is trained with data including, but not limited to, individual-provided data (experience, certifications, job history), confidence level assessments from similar individuals with comparable experience, and historical skill-confidence correlations across the user population.

The Recommendation Engine 124 is an AI model that works with the AI Chatbot Engine to determine whether to prompt additional questions for the clarification of a skill, as well as what type of question to ask. In some embodiments, the Recommendation Engine 124 is trained with data including, but not limited to, individual state information, assessment item performance histories (the Item Bank), feedback prototype questions, skill property metadata, and interaction data from prior individuals going through the assessment process.

The Skills Assessment Engine 126 is an AI model that works with the Recommendation Engine and the AI Chatbot to assess the skills of an individual. In some embodiments, the Skills Assessment Engine 126 is trained with data including, but not limited to, historical human-scored assessment responses (vectorized), scoring rubrics and Performance Level Descriptors developed by subject matter experts, validated performance benchmarks, and expert-corrected AI scores fed back into the training set over time. In some embodiments, specialized sub-models within this engine are trained on domain-specific competency data (e.g., one model for Reflective Leadership, another for AI Ethics and Evaluation).

The Job Success Fit Engine 128 is an AI model that calculates a value to determine whether an individual is a successful fit for a given job position. In some embodiments, the Job Success Fit Engine 128 is trained with data including, but not limited to, verified individual skill profiles (JobSkillsPrints), job position skill requirements, skill importance weights (determined by employers or inferred from job data), and historical performance outcomes linking individual skill profiles to job success. The model is continuously fine-tuned with updated training data from all individuals and job positions on the platform.

The Career Pathway Engine 130 is an AI model that provides an individual with recommendations to improve their skills, so they may become a successful fit for one or more job positions and advance their careers. In some embodiments, the Career Pathway Engine 130 is trained with data including, but not limited to individual skill profiles, verified proficiency distributions, job position requirements, career trajectory data, and goal-setting interactions. In some embodiments it is also trained on the outputs of the Job Success Fit Engine 128 to identify skill gaps relative to target roles.

The ML Model Training Engine 132 is a machine learning model that creates feature vectors in a Feature Vector Store 134 and weights for the various AI models, and their related features (e.g., Individual State 136, Job State 138, Skills Inferences 140, Job Titles 142, Job Descriptions 144, Skills Ontologies 146, an Item Bank 148, Rubrics and Performance Level Descriptors 150, and Feedback Prototype Questions 152). The ML Model Training Engine 132 is continuously training the Trained ML Models 105 with input from individuals and employers to provide better Skills Inferences 140, individual assessments, and job success fit calculations. In some embodiments, the ML Model Training Engine 132 is the overarching training infrastructure that creates feature vectors and weights for all the other AI models. Its inputs span essentially all data in the system: Individual State data, Job State data, Skills Inferences, Job Titles, Job Descriptions, Skills Ontologies, the Item Bank (assessment questions), Rubrics and Performance Level Descriptors, and Feedback Prototype Questions. It is continuously updated with user interactions across the platform.

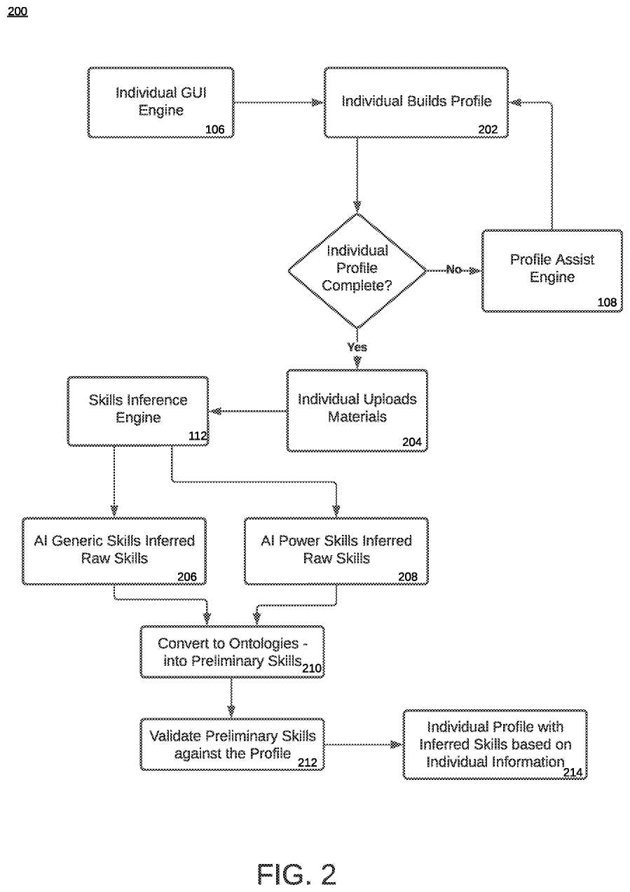

FIG. 2 illustrates a flow chart of example methods 200 for building an individual profile with individual-supplied materials. In some embodiments an individual builds a profile 202 through a Individual GUI Engine 106, by inputting biographical information, employment experience, educational history, and other relevant information. The individual may also upload digital media 204 in the form of documents, audio, video, and computer code, graphics. The Profile Assist Engine 108 aids the individual in creating a complete profile. The AI Chatbot Engine 110 may prompt the individual with questions to complete additional information. The Individual State 136 maintains information on the status of an individual's profile, and the Profile Assist Engine 108 may prompt an individual at any time to supply additional information towards completing a profile. Some fields in the individual profile may be editable by the individual, whereas other fields are static.

In some embodiments, once the Individual State 136 shows that a profile is substantially complete, a Skills Inference Engine 112 extracts skills from the individual profile. Skills may be directly input by the individual, or they may be inferred from individual information and materials. The skills may fall into two categories, Generic Skills 206 and Power Skills 208. Generic Skills are commonly referred to as core technical competencies or hard skills. These are technical skills typically required to perform a specific job or function (e.g., programming, project management, financial modeling, data analysis). Power Skills are commonly referred to as cross-functional competencies or soft skills. Power Skills are valuable across roles, teams, and industries, and often relate to how people work and collaborate (e.g., communication, collaboration, creativity, critical thinking). Power Skills are skills that are generally not considered in the talent acquisition process, such as the ability to manage different types of people, handle stressful environments, and interact with customers.

In some embodiments, the Power Skills of the Skills Ontology 146 are organized into seven core competencies that reflect human strengths in an AI-augmented economy. The organization of the Power Skills and their subskill components includes relationships between skills and subskills along with strengths of these relationships. As automation and artificial intelligence increasingly assume technical and routine tasks, the value of uniquely human capabilities, such as creativity, empathy, adaptability, leadership, and critical judgment, becomes more pronounced. These seven Power Skills enable individuals to lead, innovate, collaborate, and solve complex problems in dynamic environments. In some embodiments, Power Skills include Creative Thinking, Communication, Collaboration, Leadership, Analytical Thinking, Productivity, and AI Fluency. In some embodiments, each skill encompasses a set of subskills that further define its components, allowing for granular assessment, inference, and development through data-driven systems.

In some embodiments, Creative Thinking refers to the ability to imagine what does not yet exist and to transform abstract ideas into actionable solutions that go beyond conventional ideas and approaches, while delivering business value and/or outstanding performance. This skill involves generating novel concepts and refining them into practical outcomes through iterative development. Subskills include Divergent Thinking, which enables the generation of multiple unique responses to a challenge; Unconventional Thinking (Originality), which supports novel and non-standard approaches to problem-solving; and the ability to Evaluate and Improve Ideas, which allows for critical assessment, feedback integration, and continuous refinement of concepts to enhance feasibility, impact, or innovation.

In some embodiments, Communication is the ability to express ideas with purpose, listen deeply, and influence outcomes through shared understanding. Effective communication bridges gaps in perspective and enables alignment between individuals and teams. Subskills include the ability to Acquire Information through active listening and inquiry; Transform Information by interpreting, contextualizing, and adapting content for different purposes or audiences; and Share Information through clear, persuasive, and appropriate verbal, written, or visual expression, ensuring that messages are understood and impactful.

In some embodiments, Collaboration is the art of combining diverse perspectives, experiences, and contributions to create outcomes greater than the sum of individual efforts. It emphasizes collective progress over individual achievement and fosters inclusive, synergistic work environments. Subskills include Curiosity (Inclusion), which reflects a genuine interest in others' viewpoints and supports psychological safety; Commitment, which involves sustained engagement, accountability, and follow-through on shared responsibilities; and Leadership within group settings, which includes guiding team dynamics, facilitating equitable participation, and maintaining momentum toward common objectives.

In some embodiments, Leadership is the capacity to motivate, guide, and empower others toward shared goals, particularly in complex, uncertain, or evolving environments. It encompasses the ability to inspire action, foster accountability, and create conditions in which teams can thrive. Leadership extends beyond formal authority, emphasizing influence, vision, and the development of trust and cohesion within groups. As a Power Skill, it integrates cognitive, emotional, and strategic dimensions to enable effective guidance in collaborative and dynamic settings.

In some embodiments, Leadership includes the follow six subskills. Reflective Leadership refers to self-aware leadership characterized by the ability to understand one's strengths, blind spots, and behavioral impact; reflect on decisions; seek and act on feedback; and adapt one's approach under changing conditions with humility and learning agility. Value-driven Leadership involves making principled decisions, acting with fairness and transparency, upholding commitments, and building trust through consistent alignment between personal or organizational values and actions. Strategic Communication in Leadership is the ability to convey ideas clearly and compellingly, tailor messages to diverse audiences, listen actively, and communicate vision and strategy in ways that mobilize and align others. Relational Leadership refers to the ability to build authentic relationships, collaborate across differences, navigate conflict constructively, and influence others without relying on formal authority. Systems Thinking and Problem Framing is the ability to diagnose root causes of challenges, understand interdependencies among components, connect short-term actions to long-term impacts, and frame problems in ways that lead to productive, sustainable solutions. Innovative and Adaptive Execution is the ability to generate creative ideas, test and refine solutions through iteration, adapt to changing conditions, and drive progress amid uncertainty to achieve meaningful outcomes.

In some embodiments, Analytical Thinking is the ability to gather, interpret, and synthesize information using logic, evidence, and structured reasoning to solve complex or ambiguous problems. It supports sound judgment and decision-making in data-rich or uncertain environments. Subskills include the ability to Analyze by breaking down information into component parts to understand relationships or functions; Evaluate by assessing the credibility, relevance, and implications of evidence or arguments; Synthesize by integrating disparate data into coherent insights or models; and Extend by projecting outcomes, identifying patterns, or generating new hypotheses based on existing analysis, thereby advancing understanding beyond immediate observations.

In some embodiments, Productivity is the ability to convert knowledge, collaboration, and creativity into tangible progress, particularly in self-directed or rapidly changing environments. It reflects not only output but also the strategic management of effort, time, and learning. Subskills include Self-directed Working, which involves managing tasks, priorities, and deadlines autonomously with accountability and focus; and Self-directed Learning, which enables individuals to identify skill gaps, seek resources, and acquire new competencies independently and continuously.

In some embodiments, AI Fluency is the ability to enhance personal and professional effectiveness through the responsible, ethical, and strategic use of artificial intelligence technologies. It enables individuals to collaborate with AI as a cognitive partner and leverage intelligent tools to amplify human capabilities. Subskills include Foundational AI Tools, which involves proficiency in using AI-powered applications for content generation, data analysis, automation, and decision support; and AI Ethics and Evaluation, which encompasses the ability to assess AI outputs for bias, inaccuracy, or ethical concerns, understand model limitations, and apply human judgment when interpreting or acting on AI-generated results.

In some embodiments, each Power Skill and each of its subskills is associated with a separate and independent set of Performance Level Descriptors (PLDs). Each PLD defines a level of proficiency through specific behavioral indicators, cognitive demands, and the scope of impact relevant to that particular skill or subskill. In some embodiments, the PLDs are structured progressively, with each level reflecting increasing sophistication in application, broader influence, and greater strategic responsibility. For example, Level 1 may correspond to early career proficiency, typically demonstrated within project level contexts or individual contributions. Level 2 may reflect intermediate or management level proficiency, applied within cross functional teams or operational systems. Level 3 may represent advanced or senior executive proficiency, evidenced through enterprise-wide transformation, long term strategy, or system level change.

In some embodiments, because each Power Skill such as Creative Thinking, Communication, Leadership, Analytical Thinking, Collaboration, Productivity, or AI Fluency has its own dedicated PLD framework, the assessment of that skill is tailored to its unique dimensions and developmental trajectory. Similarly, each subskill, such as Divergent Thinking under Creative Thinking, Strategic Communication in Leadership, or AI Ethics and Evaluation under AI Fluency, is defined by its own set of PLDs, allowing for fine grained evaluation of specific capabilities independent of the parent skill. This hierarchical yet modular structure supports differentiated scoring, where an individual may demonstrate different levels of proficiency across subskills within the same overarching Power Skill.

In some embodiments, a proficiency score is assigned for each Power Skill and each subskill individually, based on alignment with the corresponding PLD, using a numerical scale such as 0 to 10. A score of 0 indicates no observable evidence of the skill or subskill, while a score of 10 indicates performance that exceeds the highest defined level of mastery. By associating discrete PLDs with both skills and subskills, the system enables precise, adaptive, and context sensitive evaluation of human competencies across roles, industries, and career stages, supporting talent development, mobility, and strategic workforce planning.

In some embodiments, the Skills Inference Engine 112 at least partially infers the Power Skills 208 and Generic Skills 206, and creates a list of skills associated with the individual. During the extraction process, a Skills Ontology Engine 114 converts the inferred skills into a standardized ontology of Preliminary Skills 210. The Preliminary Skills are then validated 212 against the information provided and stored in the individual profile 214.

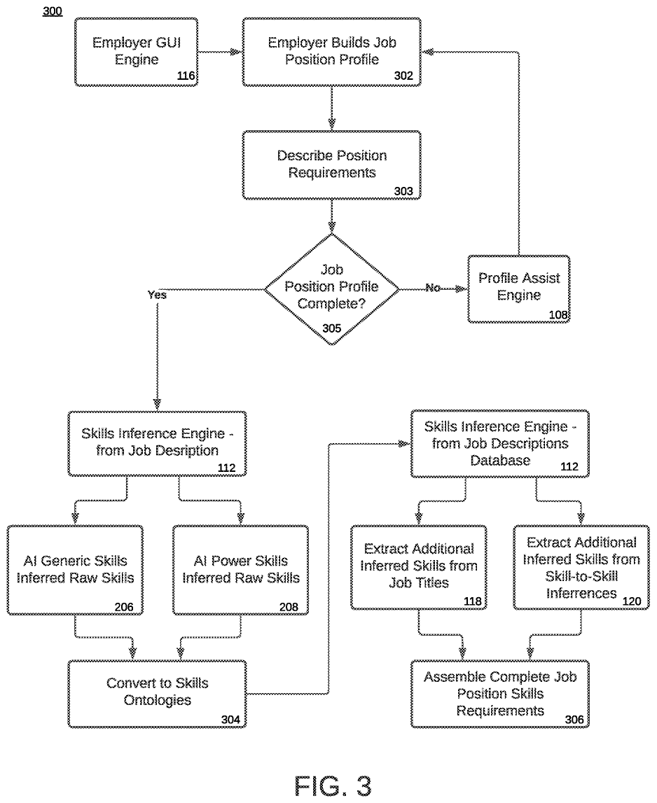

FIG. 3 illustrates a flow chart of example methods 300 for building a set of required skills for a job position. In some embodiments an employer builds a job position profile 302 through an Employer GUI Engine 116, by inputting a Job Title 142, job position information, employer information, a description of duties, a list of required skills, and other relevant information. The Profile Assist Engine 108 aids the employer in creating a complete job position profile. The AI Chatbot Engine 110 may prompt the employer with questions to complete additional information. The Job State 138 maintains information on the status of a job position profile, and the Profile Assist Engine 108 may prompt an employer at any time, whether the job position profile is complete 305, to supply additional information towards completing a job position profile.

In some embodiments, once the Job State 138 shows that a profile is substantially complete, a Skills Inference Engine 112 infers skills from the description 303 in the job position profile. Skills may be directly input by the employer, or they may be inferred from employer information provided in the job position profile. The skills may fall into the same two categories, Generic Skills 206 and Power Skills 208. The Skills Inference Engine 112 infers the Power Skills 208 and Generic Skills 206, and creates a list associated with the job position. During the extraction process, a Skills Ontology Engine 114 converts the inferred skills into a standardized ontology of skills 304.

In some embodiments, additional skills are inferred from the feature vectors provided in the Similar Job Title Engine 118 and the Skill-Skill Inference Engine 120. The Similar Job Title Engine 118 is an AI model which finds job positions with similar skills to the currently supplied Job Title 142. The model reviews similar job positions and extracts skills from those positions with appropriate weights, adding potentially appropriate Generic Skills and Power Skills that were omitted from the original Job Description 144. For example, similar Job Titles 142 may be clustered by position or industry. A similar Job Title 142 in the same industry (e.g., retail, financial services, technology) may generate heavier weighted features than the same Job Title 142 in an alternative industry. The Skill-Skill Inference Engine 120 is an AI model which generates an additional list of inferred skills. The model reviews the list of skills and extracts additional skills that are often clustered with the first list of inferred skills, adding potentially appropriate Generic Skills and Power Skills that were omitted from the original Job Description 144. A complete list of inferred skills is stored in the job position profile 302 and the Job State 138 is updated accordingly.

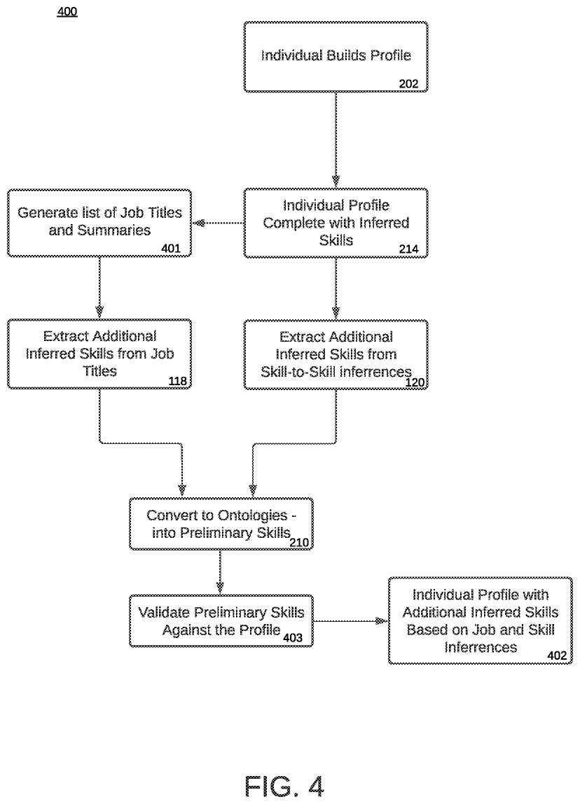

FIG. 4 illustrates a flow chart of example methods 400 for building an individual profile with inferred skills from related Job Titles 142 and associated skills. In some embodiments, an individual builds a profile 202 and the profile is supplemented with inferred skills 214. An AI model generates a list of job titles and summaries 401, based on input from the individual's profile. The Similar Job Title Engine 118 extracts skills from those positions with appropriate weights, adding potentially appropriate Generic Skills and Power Skills that were omitted from the original individual profile skills and inferred skills. Furthermore, the Skill-Skill Inference Engine 120 generates an additional list of inferred skills that are often clustered with the individual's first list of inferred skills, adding potentially appropriate Generic Skills and Power Skills that were omitted from original individual profile skills and inferred skills. During the extraction process, a Skills Ontology Engine 114 converts the inferred skills into a standardized ontology of Preliminary Skills 210. The Preliminary Skills are then validated 403 against the information provided and stored in the updated individual profile 402.

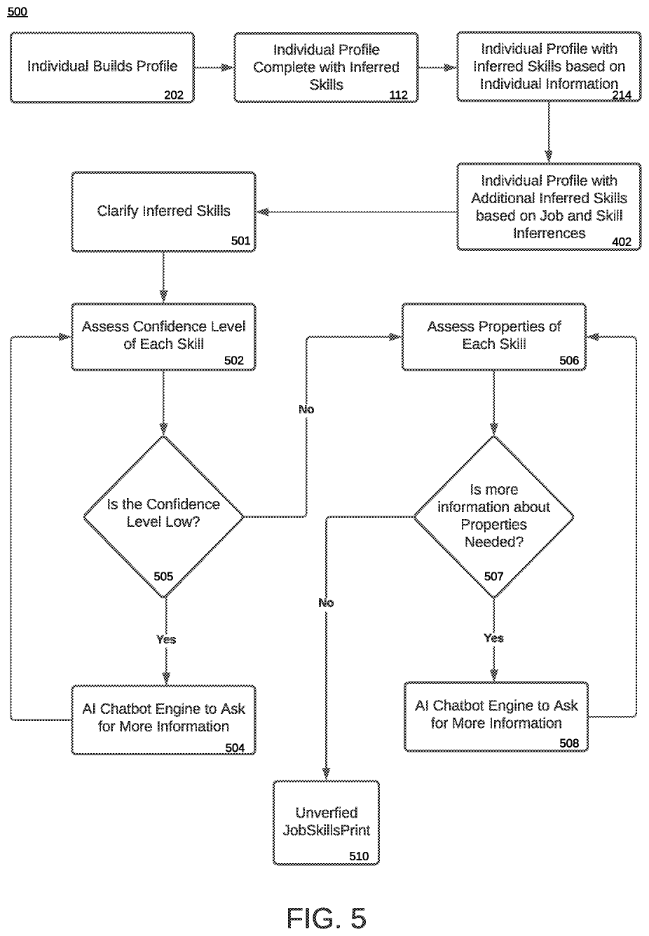

FIG. 5 illustrates a flow chart of example methods 500 for building an individual's set of unverified skills from feedback prompts. In some embodiments, the individual builds a profile 202 and then additional skills are inferred with the Skills Inference Engine 112 through the individual's information and materials 212. Additional skills are inferred through the Similar Job Title Engine 118 and the Skill-Skill Inference Engine 120 and stored in the updated individual profile 402.

In some embodiments, the talent acquisition process includes the clarification of the inferred skills 501. Each skill has associated properties. Properties may include the importance of the skill, individual's level, the number of years of experience, a reference to the information from which the skill was inferred, and the confidence level of the skill, among others. The confidence level is derived from the Confidence Level Engine 122 AI model. This model weights features associated with an individual and the associated skill using knowledge to generate a confidence level 502 for that particular individual as a function of the confidence levels for all other individuals in the trained model.

In some embodiments, the confidence level is not a reflection of the individual's skill level, but rather the model's level of confidence in the individual having that skill. For example, if a skill is inferred from other sources, but there is no information about the individual having that skill, the confidence level will be low, or zero. On the other hand, if the skill is supported by an individual having several years of experience or a certification, the confidence level may be high, or one. In some embodiments, an AI model will determine whether a confidence level is low 505. In situations where the skill is important to a job position, and the confidence level is low, the AI Chatbot Engine 110 prompts 504 the individual for additional information. After feedback from the individual, the confidence level is further assessed 502. Additional information may be requested. Once a confidence level is associated with the skill and the properties of the skill are adjusted, including the individual's skill level, the AI Chatbot Engine 110 may cease assessing the confidence level. However, at any time in the talent acquisition process, the AI Chatbot Engine 110 may prompt the individual for additional information seeking to increase a confidence level in particular skill.

In some embodiments, the properties of the skill are also assessed 506 and updated. As stated above, the properties of a skill may include the importance of the skill, individual's level, the number of years of experience, and a reference to the information from which the skill was inferred, among others. In some embodiments, an AI model will determine whether more information about the properties of the skill 507. Similar to assessing the confidence level 502, the properties of a skill are assessed using the AI Chatbot Engine 110, periodically prompting the individual 508 for additional information. The assessment of properties 506 and the assessment of confidence level 502 may occur at any time, and or simultaneously, whenever the Trained ML Models 105 determine additional individual information would be useful.

In some embodiments, the outcome of the methods to create an individual profile 200, 400, and 500, the inference of skills through the Skills Inference Engine 112, the Similar Job Title Engine 118, the Skill-Skill Inference Engine 120, and the assessment of confidence levels 502 and skill properties 506 result in an unverified JobSkillsPrint 510. This is a master set of skills, and all known information about their properties, for a given individual. The JobSkillsPrint 510 is an evergreen entity and is continuously being updated by the various AI models as they are trained with information across all individuals and job positions.

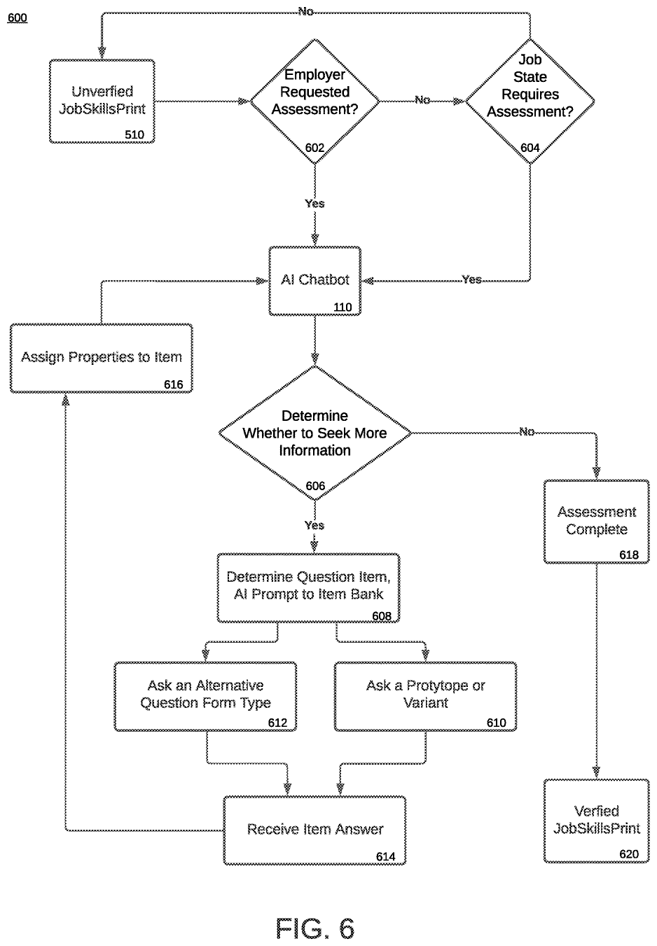

FIG. 6 illustrates a flow chart of example methods 600 for assessing and verifying an individual's set of skills through question prompts. In some embodiments, an employer 602 or a Job State 138 may request 604 an assessment of an individual's skills. The Skills Assessment Engine 126 prompts the AI Chatbot Engine 110 to perform the assessment by interacting with the Individual GUI Engine 106 to ask the individual one or more questions. A Recommendation Engine 124 determines whether to seek more information 606 to assess one or more of an individual's skills. If information is sought, the AI Chatbot 110 prompts the Item Bank 148 for questions 608 to ask the individual in order to assess the skills. The Item Bank 148 is a dynamic repository of questions related to various skills. The Item Bank 148 has a set of Feedback Prototype Questions 152 to ask an individual about a particular skill. An example open-ended Feedback Prototype Question 152 could be, “How many years have you had experience at the company?” The Recommendation Engine 124 may prompt the AI Chatbot Engine 110 to ask the Feedback Prototype Question 152, or in a variation of the question, to introduce variety 610. If a variation is created, the new variation may be stored in the Item Bank 148 for future use.

In some embodiments, the Recommendation Engine 124 may determine that an alternative question type would be appropriate 612. Alternative question types may include binary questions, multiple choice questions, ranking questions, etc. For example, the Recommendation Engine 124 may prompt the AI Chatbot Engine 110 to ask an open-ended Feedback Prototype Question 152, “How do you typically generate new ideas?” Alternatively, the Recommendation Engine 124 may prompt the AI Chatbot Engine 110 to ask a ranking question. In this example, a statement may be presented, “I frequently try out new ideas, even if they might fail.”, and the individual is requested to move a slider somewhere between “Strongly Agree”, and “Strongly Disagree”.

In some embodiments, when an answer is received 614, properties of the skill are assigned or updated 616. These properties are stored in the Item Bank 148. Answers may have a positive or negative impact on properties of the skill. In some embodiments, an incorrect answer may lower an individual's skill level property, whereas a correct answer may increase the individual's skill level property. Some answers may generate the Recommendation Engine 124 to prompt the AI Chatbot Engine 110 to ask a follow up question related to the same skill. Other answers may complete the inquiry, and the Recommendation Engine 124 will cease that line of questioning, satisfied with the assessment of that particular skill.

In some embodiments, a Recommendation Engine 124, implemented as an AI model, selects and delivers a Feedback Prototype Question 152, also called an Assessment Item, to an individual through the Individual GUI Engine 106. In some embodiments, the Recommendation Engine 124 tailors each Assessment Item to the individual based on their profile, prior interactions, current role, targeted job level, and developmental objectives. In some embodiments, the Recommendation Engine 124 creates one or more entirely new Assessment Items based on information about the individual. In other embodiments, the Recommendation Engine 124 generates a custom Assessment Item for one or more Power Skills and/or underlying subskills based on the Skills Ontology 146 and additional information about an organization, employer, or cohort of individuals. This personalization ensures that the assessment is contextually relevant and appropriately challenging, whether used for evaluating readiness for a current position or preparing for a future role. The Assessment Item is designed to elicit observable evidence of one or more Power Skills and/or underlying subskills, enabling granular inference by the Skills Assessment Engine 126.

In some embodiments, an Assessment Item is grounded in realistic workplace scenarios to reflect the complexity of professional decision making. Examples include responding to a video clip depicting a team conflict, drafting a reply to a challenging email, interpreting a direct message from a manager, or summarizing action steps after a meeting summary. Each item presents a situation that requires the individual to make tradeoffs, prioritize competing demands, or exercise judgment under pressure. Responses are typically open ended and constructed, such as written explanations, proposed strategies, or drafted communications, allowing individuals to demonstrate depth of thinking.

In some embodiments, the Assessment Item uses an ordered multiple-choice format, where answer options are arranged along a continuum of quality, effectiveness, or sophistication, enabling assessment of nuanced reasoning rather than binary correctness. In some embodiments, a time limit, such as 10, 15, or 20 minutes, is associated with each item, with duration and complexity calibrated according to the expected proficiency level of the target role. For example, Level 1 items may focus on project level decisions with shorter response windows, while Level 3 executive items may involve strategic dilemmas requiring deeper analysis and longer response times. Conversely, an Assessment Item may allot additional time to answer for a Level 1 user than for a Level 3 user.

In some embodiments, the AI Chatbot Engine 110 dynamically generates the Assessment Item, creating a scenario that reflects current organizational challenges or role specific demands. When generated by AI, the system also identifies which Power Skills and subskills are embedded in the scenario, using semantic analysis to align the item with the skill taxonomy.

In some embodiments, each Assessment Item is associated with structured metadata that defines its purpose, structure, and evaluation logic. The metadata specifies which of the seven Power Skills, such as Communication, Leadership, or AI Fluency, are assessed by the item, as well as the specific subskills involved, such as Relational Leadership or Evaluate and Improve Ideas. In some embodiments, skills and subskills are organized in a hierarchical structure extending across multiple levels, including deep or infinite taxonomies, or represented as a network of correlated competencies where relationships between skills, such as between Analytical Thinking and Systems Thinking, are modeled to support cross skill inference.

In some embodiments, the metadata also includes a scoring methodology to be applied, which varies depending on the item type. For binary recognition tasks, a simple 0/1 scoring model may be used. For constructed responses, more sophisticated methods apply, such as rubric based scoring, semantic similarity matching to exemplar answers, or AI driven evaluation. The metadata further indicates whether the item is targeted to the individual's current job level or a desired future role, supporting both diagnostic and developmental use cases. Additionally, in some embodiments, the metadata associates dedicated AI models with specific subskills, for example, a natural language model trained on reflective discourse may evaluate responses for evidence of Reflective Leadership, while another model focused on ethical reasoning may assess AI Ethics and Evaluation. These subskill specific models analyze the individual's response and generate proficiency signals that are used to update skill properties in the system.

In some embodiments, if no further information is sought by the Recommendation Engine 124, the assessment is complete 618 and a verified JobSkillsPrint is created 620. The Individual State 136 is updated to reflect a completed assessment of particular skills. At any time, the Skills Assessment Engine 126 may prompt the AI Chatbot Engine 110 to perform another assessment of the same skills or a new skill. In some embodiments, a skill may be verified automatically if there is concrete evidence of the skill, such as a certification, without questioning the individual.

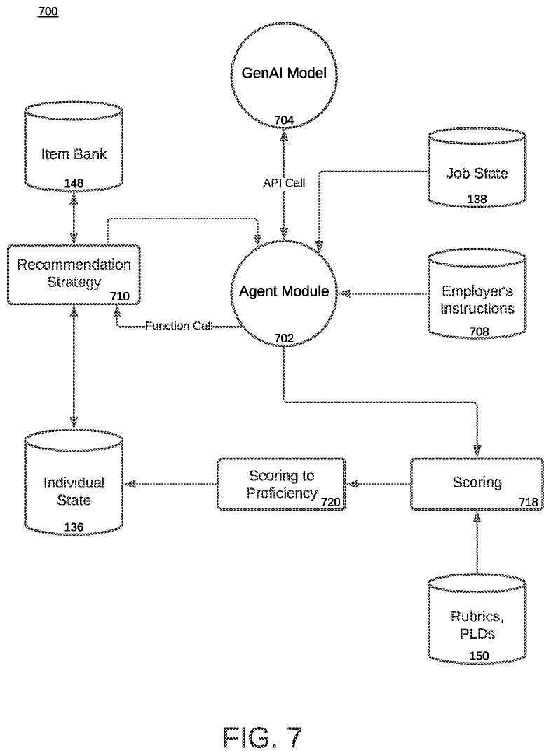

FIG. 7 is a data flow diagram of an example process 700 for assessing and verifying an individual's set of skills through question prompts. In some embodiments, an Agent Module 702 controls all process engines, including the Individual GUI Engine 106, the Profile Assist Engine 108, the AI Chatbot Engine 110, the Skills Inference Engine 112, the Skills Ontology Engine 114, the Employer GUI Engine 116, the Similar Job Title Engine 118, the Skill-Skill Inference Engine 120, the Confidence Level Engine 122, the Recommendation Engine 124, the Skills Assessment Engine 126, the Job Success Fit Engine 128, the Career Pathway Engine 130, and the ML Model Training Engine 132, among others.

The Agent Module 702 interacts with one or more generative AI models 704 through an Application Programming Interface (API). Generative AI models include models for inferring skills from individual-provided materials, inferring skills from other skills, inferring skills from job positions, prompting the employer interface, prompting the individual interface, building the individual profile, building the job position profile, recommending assessment questions, recommending confidence level questions, and recommending properties assessment questions among others.

In some embodiments, the Job State 138 stores information about the status of a job position. Status information includes the job position's skills, the Job Title 142, the Job Description 144, inferred skills, information about whether the job position is filled, and potential individuals suitable for the job position. Employer's instructions 708 inform the Agent Module 702 with input regarding the job position, and the employer's desires for potential individuals during the talent acquisition process.

In some embodiments, the Agent Module 702 calls the Recommendation Engine 124 to prompt a Recommendation Strategy 710 towards the goal of assessing one or more skills of an individual. An Item Bank 148 stores feedback prototype questions and assessment items for prompting feedback from the individuals and/or employers. In some embodiments, the Item Bank 148 stores property information of each skill associated with the individuals and/or employers. The Recommendation Engine 124 prompts questions from the Item Bank 148 and may add questions to the Item Bank 148 as variations and new questions are created by the Skills Assessment Engine 126 during assessment. These new questions are generated in real-time through the tuning of the various Trained ML Models 105 supporting the AI Chatbot Engine 110 and the Recommendation Engine 124.

In some embodiments, the Recommendation Engine 124 informs the Individual State 136 of various information generated from feedback. During assessment 600 and verification of an individual's set of skills through question prompts, the Agent Module 702 may reference scoring Rubrics and Performance Level Descriptions 150 to aid in the automatic scoring 718 an individual's skill level. Initial Rubrics 150 may be developed manually by an employer, or they may be trained on the Skills Assessment Engine 126 AI model over time, as additional individuals are assessed. In some embodiments, the Rubrics and PLDs 150 are continuously updated with additional information. An individual's skill level may be automatically scored as a level of proficiency 720, such as beginner, intermediate, or expert, as a percentage of expertise, or through any other scoring system.

In some embodiments, Assessment Items are scored to evaluate an individual's proficiency in one or more Power Skills or subskills. All scores are skill specific, reflecting performance relative to the defined expectations for that particular skill or subskill. An Assessment Item may relate to multiple skills or subskills in varying degrees. In some embodiments, an individual's response is scored for a given skill or subskill, the resulting proficiency propagates through the hierarchical or correlated skill network, updating related skill assessments to improve overall accuracy and confidence in the skill profile. This propagation allows evidence from a single response to inform multiple related competencies, enabling more efficient and coherent assessment.

In some embodiments, Assessment Items are evaluated using a Scoring Rubric 150, which provides structured criteria for assigning scores based on reference data derived from expert analysis, historical responses, or validated performance benchmarks. The Scoring Rubric 150, in conjunction with the targeted skills and subskills, determines which AI scoring model is used to analyze the response. Different rubrics may route assessment data to specialized AI models trained to recognize specific competencies, such as Reflective Leadership or AI Ethics and Evaluation. Some Scoring Rubrics 150 are initially developed by subject matter experts and expressed in plain language, describing the behavioral or cognitive indicators associated with each numeric score level. Over time, these rubrics may be refined through machine learning to enhance precision and consistency in scoring.

In some embodiments, individual scores derived from Assessment Items are treated as signals. These signals are measurable inputs or pieces of evidence that serve as inputs for estimating an individual's proficiency in a given Power Skill or subskill. A score is not the same as a proficiency estimation. Instead, it functions as an observed data point that contributes to a probabilistic inference process. Each score is assigned a weight based on metadata associated with the Assessment Item, such as its difficulty level, response format, relevance to the target skill, or the recency of the response. Scores from more recent or higher fidelity assessments may be assigned greater weight to reflect their increased reliability and predictive value.

In some embodiments, proficiency in a skill or subskill is modeled as a latent variable. A latent variable is a trait that cannot be directly observed but is inferred from observable behaviors and responses. In some embodiments, proficiency is represented as a probability distribution, such as a normal distribution defined by a mean and a standard deviation. The mean represents the current best estimate of the individual's skill level. The standard deviation represents the uncertainty in that estimate and defines a credibility interval around the mean. In one embodiment, this proficiency estimate is computed using Bayesian statistical methods. In this approach, a prior distribution reflecting the initial belief about the individual's skill level is updated using new evidence to produce a posterior distribution that reflects the revised belief.

In some embodiments, a Confidence Level is associated with each proficiency estimate and indicates the degree of certainty in the current assessment. The Confidence Level is informed by the individual's own response patterns, including consistency and coherence across assessments. It is also informed by data from other individuals who have completed the same or related Assessment Items. This aggregate data helps calibrate the interpretation of individual responses and improves the robustness of the inference. As additional evidence is collected, the posterior distribution typically becomes narrower, indicating increased confidence in the estimate. In some cases, however, responses that vary significantly from expected patterns may increase uncertainty, leading to a broader distribution and a lower Confidence Level. Such outcomes may trigger additional assessment to resolve ambiguity.

In some embodiments, evidence used to estimate proficiency is accumulated through three primary pathways. First, the number of Assessment Items administered to the individual can be increased, providing more data points over time. Second, multi-tagged Assessment Items that are associated with multiple skills or subskills can be used to generate simultaneous signals across related competencies. Third, unstructured data sources provided by the individual, such as resumes, cover letters, project descriptions, or other uploaded materials, can be analyzed to supplement the assessment and provide additional context. In some embodiments, the unstructured data sources may include employer materials, such as business documents, meeting notes, and recordings. The quality, variety, and cognitive complexity of Assessment Items directly influence the system's ability to discriminate between different levels of proficiency. A broader range of well-designed items enables finer grained assessment and supports higher confidence in the resulting proficiency estimates.

In some embodiments, the scoring of Assessment Items involves artificial intelligence, depending on the item type and associated metadata. For Assessment Items with structured response formats, such as binary 0/1, multiple choice, or ranking tasks, scoring may be performed without AI, using rule-based logic or direct comparison to predefined correct answers. However, for items requiring interpretation of open ended, constructed responses, such as written explanations, strategic recommendations, or communication drafts, AI is employed to generate a proficiency signal. The use of AI is determined by the scoring method specified in the Assessment Item's metadata, which defines the appropriate evaluation approach based on format, complexity, and targeted skills.

When AI is used, in some embodiments, scoring is a function of three key inputs: the individual's response, the Assessment Item's metadata, and the associated Scoring Rubric 150. These inputs are jointly processed by an AI scoring model specifically selected or configured for the task. In some embodiments, the AI model is a fine-tuned machine learning model trained on a historical dataset of human scored responses. This training data is vectorized, enabling the model to learn patterns of language, reasoning, and structure that correlate with different proficiency levels. Similarly, the content of the Assessment Item and the individual's response are vectorized and encoded into numerical representations that the model can process.

In some embodiments, the AI scoring process employs a Retrieval Augmented Generation pipeline, in which the system retrieves instructive example answers, such as high quality, expert validated responses, from a reference database and uses them to inform the scoring decision. These examples serve as contextual prompts, improving the model's ability to align its output with human judgment.

In some embodiments, the AI model generates both a score and a rationale explaining the basis for that score, such as identifying evidence of critical thinking, leadership principles, or ethical reasoning present in the response. This rationale enhances transparency and supports downstream review.

In some embodiments, multiple AI scoring engines operate in parallel, each specializing in a particular Power Skill or subskill, such as Reflective Leadership, Analytical Thinking, or AI Ethics and Evaluation. Each engine evaluates the response independently, producing a skill specific score and accompanying rationale. These individual outputs are then aggregated to form a composite assessment of the individual's performance across multiple competencies. This parallel architecture improves accuracy and modularity, allowing for targeted model updates and reducing error propagation compared to serial processing.

In some embodiments, human subject matter experts may review and validate AI generated scores and rationales. In cases where a human disagrees with the AI output, the expert may override or replace the machine generated score. The corrected response, along with the expert rationale, is stored in the historical data repository, further enriching the training set and enabling continuous improvement of the AI models. This feedback loop ensures that the system evolves with real world insights and maintains alignment with professional standards.

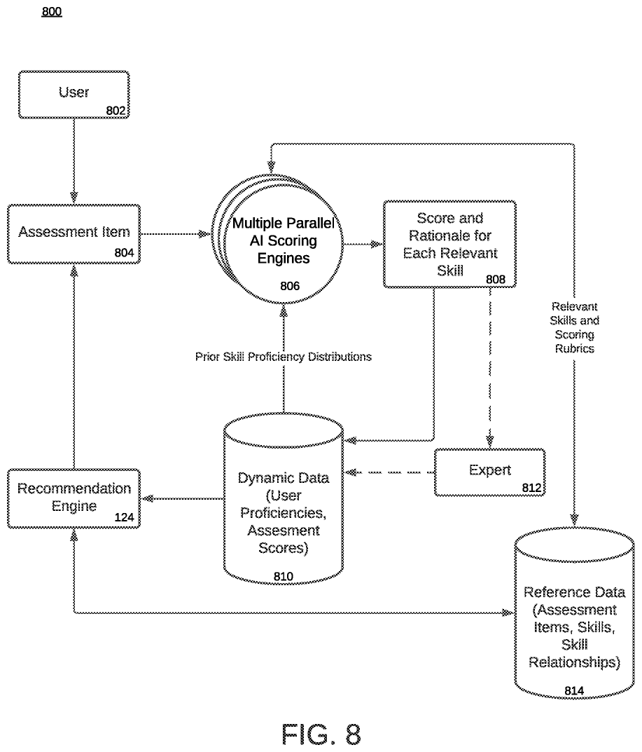

FIG. 8 is a data flow diagram of an example process 800 for assessing skill proficiency. In some embodiments, a user 802 provides input in response to an Assessment Item 804. The user's answer is then scored by one or more AI scoring engines 806 operating in parallel. In an instance where the Assessment Item 804 is comprised of multiple skills or subskills, a separate AI model scores each skill or subskill independently. In some embodiments, each score is also accompanied by a rationale 808, explaining why the reasoning for the score of the relevant skill. The score and rationale 808 are then stored in a data repository 810 along with other dynamic data regarding the user. In one embodiment, dynamic data includes user skill proficiency distributions in skills and subskills, and the underlying assessment scores. These proficiency distributions inform the AI scoring engines 806 in the calculation of scores for future Assessment Items. In an alternative embodiment, an expert 812 reviews the answer to the Assessment Item 804, along with the AI scoring engine's score and rationale. The expert 812 may agree with the score as it is or modify the score and rationale for inclusion in the data repository 810.

In some embodiments, a second set of reference data 814 provides a hierarchy of skills, correlation of skills, and scoring rubrics. This reference data provides the AI scoring engines 806 with data to calculate scores and rationales of Assessment Items. The skills, and skill relationships, provide a Skills Ontology 146 comprising a hierarchy of skills and subskills, linking one skill to another. Some skills are dependent on others, and some skills share a correlation but are not strictly hierarchical. Relationships between skills carry a weight, determining the influence that the assessment of one skill or subskill has on the assessment of another skill or subskill. This network of relationships between skills provides a framework for updating multiple skills assessments from a single Assessment Item response. When a user receives a score for an Assessment Item 804, the skills or subskills measured in that assessment item are updated and recorded in the dynamic data repository 810. Likewise, the related skills and subskills of hierarchies or correlations are updated.

In some embodiments, a Recommendation Engine 124 accesses the dynamic data repository and the reference data to determine which assessment item to provide to the user.

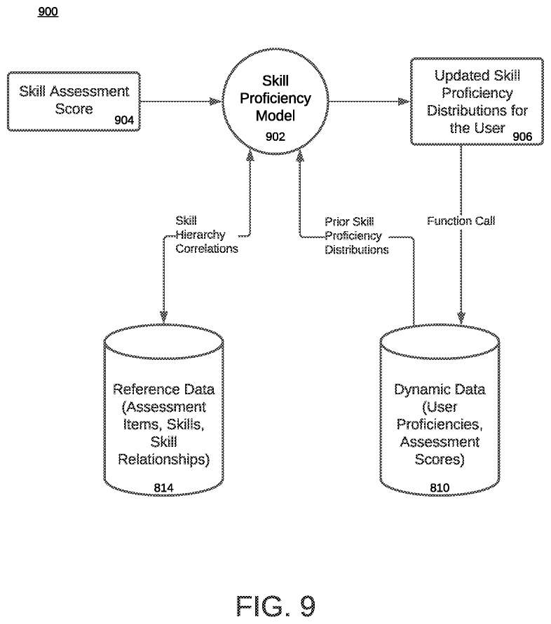

FIG. 9 is a data flow diagram of an example process 900 for updating skill proficiency distributions. As discussed above, in some embodiments, reference data 814 and dynamic data 810 provide data to the AI scoring engines 806 for the calculation of scores of Assessment Items 804. These two data repositories provide the same data to an AI Skill Proficiency Model 902 for the calculation of skill proficiencies. Skill proficiencies are latent variables, represented by probability distributions. The means of the probability distributions are approximations of the skill proficiencies, but the true proficiency is latent. Therefore, each distribution has a standard deviation, or confidence level, which indicates the accuracy of the mean. In some embodiments, the AI Skill Proficiency Model 902 is trained with data including, but not limited to, assessment item scores, reference data comprising skill hierarchies and correlations (the Skills Ontology), scoring rubrics, and dynamic user data including prior proficiency distributions and response patterns.

In some embodiments, a skill assessment score is sent 904 to the Skill Proficiency Model 902, which calculates the proficiency distribution. As additional assessment scores are provided, the proficiency distributions for the user are updated 906. The assessing of additional scores tends to improve the accuracy of the distribution. As proficiency distributions are updated, they likewise update the dynamic data repository.

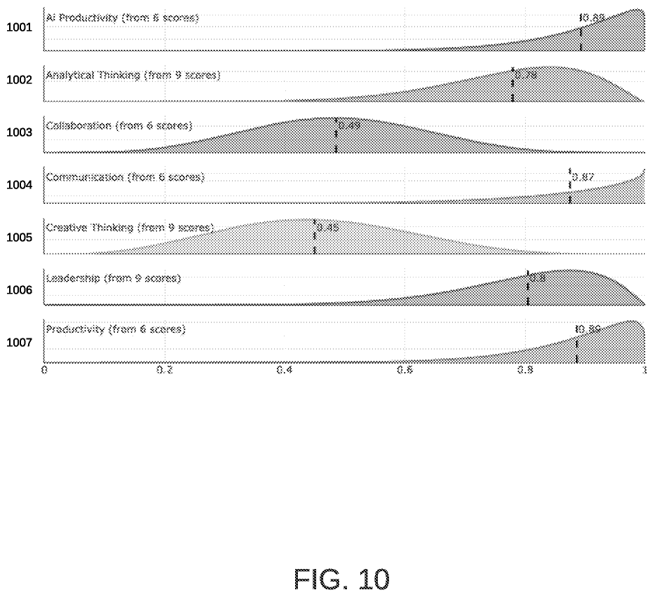

FIG. 10 shows seven example skill proficiency distributions. In some embodiments, the distributions are bounded [0,1]. The first curve 1001 shows a skill proficiency distribution for AI Productivity based on six scores, with a monotonic increase towards 1 and a mean of 0.89. This curve shows evidence of a very high proficiency. The second curve 1002 shows a skill proficiency distribution for Analytical Thinking based on nine scores, with a unimodal peak near 0.8, and a mean of 0.78. This curve shows evidence of a high proficiency. The third curve 1003 shows a skill proficiency distribution for Collaboration based on six scores, with a symmetric bell centered around a mean of 0.49. This curve shows evidence of mid-level proficiency. The fourth curve 1004 shows a skill proficiency distribution for Communication based on six scores, with a sharp spike near 1 and a mean of 0.87. This curve shows near-certain mastery. The fifth curve 1005 shows a skill proficiency distribution for Creative Thinking based on nine scores, with a moderate symmetric bell centered around a mean of 0.45. This curve shows an average and uncertain skill estimate. The sixth curve 1006 shows a skill proficiency distribution for Leadership based on nine scores, with a broad distribution around 0.8. This curve shows good proficiency but more uncertainty than the similar second curve. The seventh curve 1007 shows a skill proficiency distribution for Productivity based on six scores, with a mass heavily concentrated close to 1 and a mean of 0.89. This curve shows high proficiency and strong confidence.

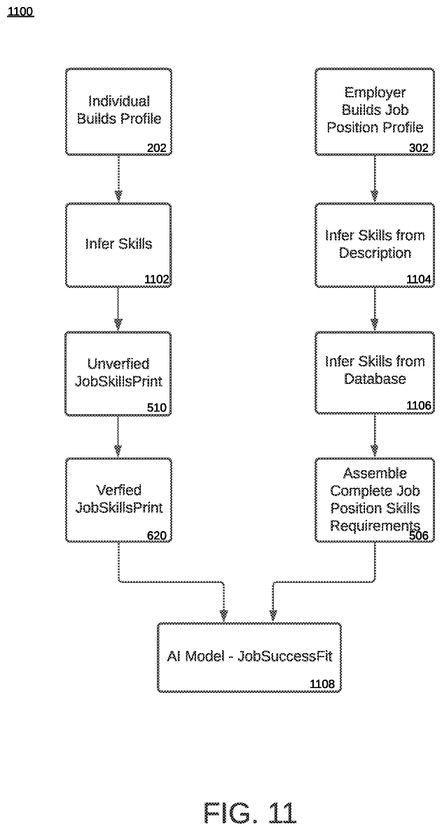

FIG. 11 illustrates a flow chart of example methods 1100 for calculating an individual's successful fit with a job position. In some embodiments, the individual builds a profile 202 as described in the method 200, and skills are inferred 1102 through the individual's materials, from other associated skills, and from Job Titles 142 to form an unverified JobSkillsPrint 510 as described in the method 500. The Skills Assessment Engine 126 assesses one or more skills of the individual and creates a verified JobSkillsPrint 620. In a parallel process, the employer builds a job position profile 302 as described in the method 300. Skills are inferred 1104 from the Job Description 144, similar Job Titles 142 and associated skills 1106. A complete list of inferred skills 506 is stored in the job position profile 302. The Job Success Fit Engine 128 then automatically calculates a score on how well a particular individual would fit a particular job position. In some embodiments, a JobSuccessFit score 1108 is available for every individual in relation to every job position. The JobSuccessFit score 1108 may be an integer on a scale, or a percentage, or any other scoring system. Employers may see a list of the highest scoring individuals and then seek to acquire additional information from the individuals through further skill assessment or clarification of skills. In some embodiments, individuals may see a list of their highest scoring job positions.

In some embodiments, the JobSuccessFit score 1108 is calculated using a generalized algorithm that models fit as a function of verified skill proficiencies, job requirements, and skill importance weights. The algorithm operates on the principle that fit is a latent variable, unobserved but inferred from individual and job data, and is reported as an expected value derived from probabilistic skill estimates.

In some embodiments, for a given skill s, let xₛ represent the individual's proficiency in that skill, modeled as a random variable with a posterior distribution obtained from prior assessments, responses, and evidence aggregation as described in earlier sections. Let L(x) be a link function that maps a proficiency level x to a fit value in the interval [0,1], representing the degree to which the proficiency meets the job's requirement. In one embodiment, the link function is defined as L(x)=min(x/X, 1), where X is the threshold level of proficiency required by the job for skill s. This formulation ensures that fit increases linearly with proficiency up to the threshold, after which it saturates at 1, indicating full adequacy.

In some embodiments, the expected fit for skill s is then computed as the expectation of the link function with respect to the posterior distribution of xₛ, denoted: fₛ=E_post[L(xₛ)]. This expectation integrates over the uncertainty in the individual's proficiency, yielding a skill specific fit value fₛ in [0,1] that reflects both the estimated level and confidence in that estimate.

Each individual's overall fit to a job is characterized by a set of skill specific fit values {fₛ∈[0,1], s=1, 2, 3, . . . }, one for each relevant Power Skill or subskill. These values may be aggregated into a single scalar score, referred to as the JobSuccessFit score, using a weighted average: JobSuccessFit=Σwₛ·fₛ/Σwₛ where wₛ is a non-negative weight parameter associated with skill s in the job position profile 302. These weights reflect the relative importance of each skill to success in the role and are determined by the employer, inferred from job data, or learned from historical performance outcomes.

In some embodiments, individuals are ranked based on this aggregate JobSuccessFit score to produce a primary list of top matches. Additionally, a Pareto optimality analysis may be performed to identify individuals who are superior in all assessed skills compared to others, i.e., for whom fₛ≥fₛ across all skills s, with strict inequality for at least one. Such Pareto optimal individuals are added to a shortlist, as they represent unambiguous high fit individuals. For the remaining individuals, a secondary or wait list is generated by ranking based on the scalarized JobSuccessFit score. This two-tiered approach enables employers to quickly identify both dominant individuals and those with strong overall alignment, even if not optimal across every dimension.

Additionally, individuals may see their desired job positions and where they may lack and/or need to improve skills. In some situations, the individual may realize they have additional skills desired by the employer, and may add them to their profile, or acquire them and add them in the future.

In some embodiments, an Action Plan is generated and maintained by an AI model that creates a tailored development strategy directly linked to an individual's assessment responses, verified skill profile, and target job requirements. The Action Plan is not a static list of recommendations but a dynamic, adaptive framework that evolves as the individual progresses, receives new feedback, or updates their goals. As an AI-driven component of the Career Pathway Engine 130, the Action Plan provides continuous, context-aware guidance to support long-term skill growth.

In some embodiments, the Action Plan includes specific, actionable items designed to develop individual Power Skills and subskills if the individual's current proficiency is below a defined threshold, or even in those areas has not yet been assessed. This ensures that development is proactive and comprehensive, rather than limited to remediation of identified gaps. Each action item is calibrated to the individual's current level, learning pace, and work context, enabling meaningful engagement without overwhelming the user.

In some embodiments, a feature of the Action Plan is the inclusion of micro exercises, small, focused activities that can be performed throughout the day during regular work or personal routines. Examples include prompts such as “Ask one sharper question in your next meeting,” “Paraphrase a colleague's point before responding,” or “Evaluate an AI-generated output for potential bias.” These micro exercises are designed to build habits through deliberate practice, embedding skill development into everyday behavior. Over time, repeated performance of these actions reinforces cognitive and behavioral patterns associated with high proficiency.

In some embodiments, the Action Plan functions as a virtual coaching tool, providing structured support for talent development. Individuals can use the plan to train themselves independently, track progress, and reflect on outcomes. In some embodiments, completion of action items triggers follow-up assessments or recognition within the system, closing the loop between practice and validation. Employers may use the Action Plan to guide employee development, aligning individual growth with team and organizational objectives.

In some embodiments, the Career Pathway Engine 130 provides an individual with recommendations to improve their skills. It may be useful to an independent individual, so they may become a successful fit for one or more job positions. It may also be a useful tool for employers, as it can guide their current employees in the development of skills, boosting their performance and benefiting the company. The Career Pathway Engine 130 can create personalized progression opportunities for an employee's current role but also illuminate opportunities for growth in alternative roles, which match their developing skill set.

Employers may use the Career Pathway Engine 130 to develop their current employees and enhance employee retention.

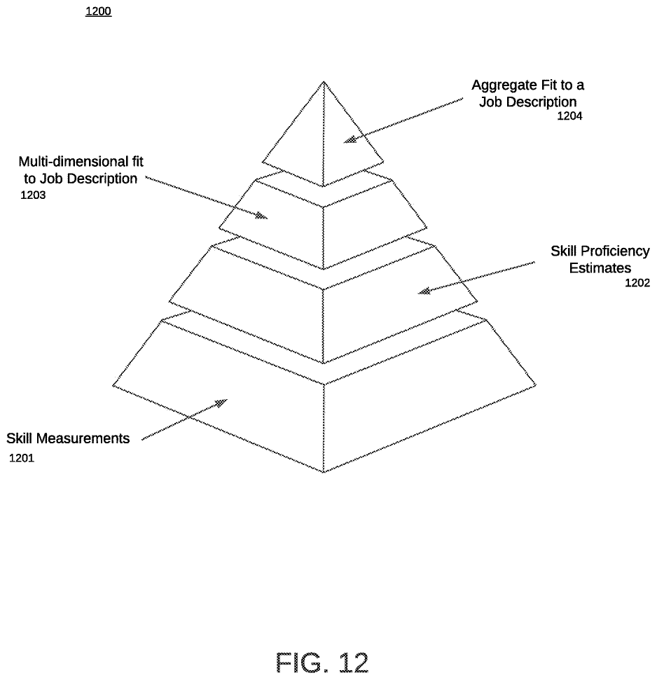

FIG. 12 shows a conceptual diagram of a pyramid, demonstrating one embodiment for a process 1200 of determining an individual's fit to a job position. In some embodiments, the disclosed system evaluates individual suitability for a position through a multi-stage process that incrementally transforms information into an aggregate compatibility score. In a first step 1201, the system measures skills by collecting structured and unstructured evidence associated with an individual, including resumes, work history, project artifacts, assessment responses, behavioral signals, and interaction data, and extracting observable skill indicators using automated feature extraction, natural language processing, and adaptive assessment techniques. In a second step 1202, the system estimates skill proficiencies by converting the measured indicators into quantitative proficiency values for each skill dimension, for example by applying probabilistic inference models that generate posterior distributions or confidence-weighted scores representing both estimated ability and associated uncertainty. In a third step 1203, the system receives a multi-dimensional fit to a job description's various skills by comparing the individual's proficiency vector to a corresponding job skill requirement vector, computing per-skill compatibility measures or distance metrics across the plurality of skill dimensions. In a fourth step 1204, the system receives an aggregate fit to the job description by combining the per-skill compatibility measures using a weighted aggregation function, predictive model, or scoring algorithm to produce a unified JobSuccessFit score 1108 that represents overall expected suitability of the individual for the position.

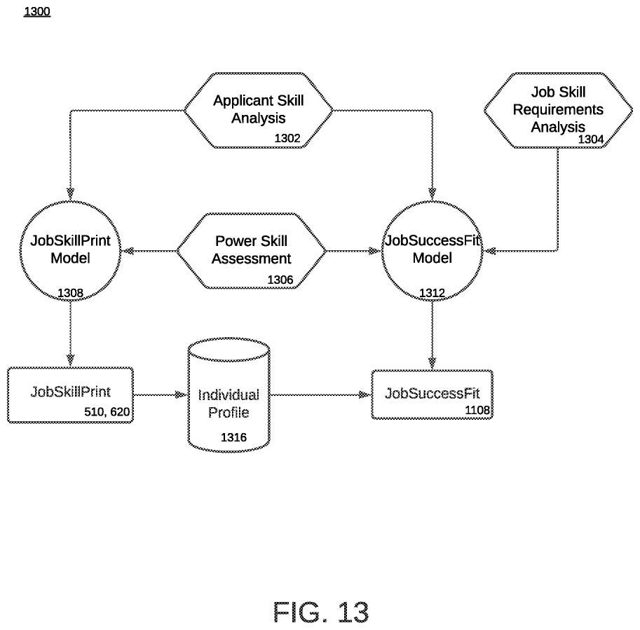

FIG. 13 is a high-level data flow diagram of an example process 1300 calculating an individual's successful fit with a job position. In some embodiments, the process may be divided into three main categories, the analysis of applicant skills 1302, the analysis of job requirement skills 1304, and the assessment of an individual's Power Skills 1306. The individual's skills are inferred through the Skills Inference Engine 112, the Similar Job Title Engine 118, and the Skill-Skill Inference Engine 120. These skills are assembled by the JobSkillsPrint Model 1308 into an unverified JobSkillsPrint 510, and stored in the individual's profile 1316. The Skills Assessment Engine 126 assesses the individual's unverified JobSkillsPrint 510, creating a verified JobSkillsPrint 620, of an individual's Power Skills and Generic Skills. In some embodiments, the JobSkillsPrint Model 1308 is trained with data including, but not limited to, individual profiles, inferred skills outputs from the other engines, and job position requirement data.

In some embodiments, the job position's required skills are inferred through the same engines, the Skills Inference Engine 112, the Similar Job Title Engine 118, and the Skill-Skill Inference Engine 120, and assembled into a complete list of requirements. A JobSuccessFit Model 1312 automatically calculates a score, the JobSuccessFit 1108, for how well an individual would fit a particular job position, comparing the individual's verified JobSkillsPrint 620 and jobs position requirements. The JobSuccessFit 1108 scores are associated with the individual's profile and the job position profile, and are subject to automatic continuous recalculation as the JobSuccessFit Model 1312 is fine-tuned with additional information regarding the particular individual and job position, as well as the feature weights generated from constantly updated training data from all individuals and job positions.

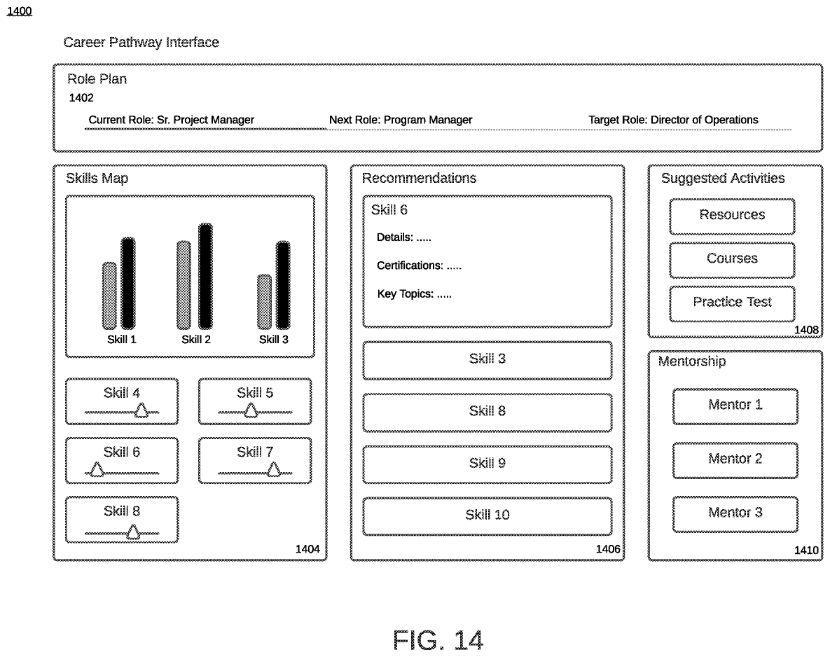

In some embodiments, a Career Pathway Interface 1400 is accessible through the Individual GUI Engine 106 and/or the Employer GUI Engine 116. See FIG. 14. This interface showcases the skills of an individual or employee and provides recommendations on how to progress in their careers. One display element may include a Role Plan 1402, which states an employee's current role, a target role, and an intermediate role on the path to the target role. To attain career advancement, another element of the interface may provide detailed assessments of the employee's Generic Skills 206 and Power Skills 208 in a JobSkillsPrint 510. A Skills Map 1404 may reflect these skills in bar charts or sliders, along with projections for what skill level improvements would be desirable to advance to the next role.

In some embodiments, the Career Pathway Interface is further comprised of Recommendations 1406 of new skills to acquire. These skills may be determined by the Career Pathway Engine 130, and list certifications and details about the skills. Another element may provide Suggested Activities 1408, including educational resources to hone or acquire skills. Some embodiments may include a list of mentors 1410 to coach the employee in developing the recommended skills to achieve career advancement.

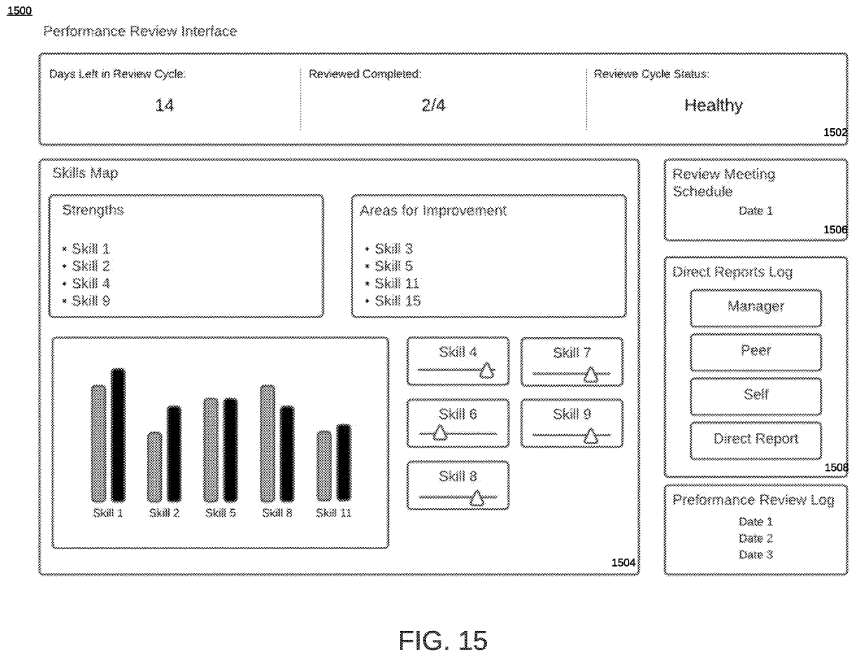

In some embodiments, a Performance Review Interface 1500 is accessible through the Individual GUI Engine 106 and/or the Employer GUI Engine 116. See FIG. 15. This interface provides details about an employee's skills, as reflected in performance reviews and the JobSkillsPrint 510, as assessed by the Skills Assessment Engine 126. One element of the interface may include an overall summary of the performance review cycle 1502, with details about days left in the cycle, how many reviews have been completed, and the overall status of the cycle. A Skills Map 1504 may reflect the employee's Generic Skills and Power Skills in bar charts or sliders, as assessed by human reviewers or according to the Skills Assessment Engine 126. Strengths and areas for improvement with relation to the employee's current role are listed.

In some embodiments, the Performance Review Interface 1500 is further comprised of information regarding review meetings 1506, including the dates of the next scheduled review and past reviews. In addition to the Skills Assessment Engine 126, skills may be assessed in performance reviews 1508 by various individuals. These include Managers, Peers, Direct Reports, and the employee themselves.

In some embodiments, the systems and methods described herein are applied across multiple talent management contexts to enhance career advancement readiness, improve placement accuracy, and strengthen alignment between individual capabilities and job expectations. By integrating automated skills inference, adaptive assessment, probabilistic proficiency estimation, JobSuccessFit 1108 calculation, and personalized development planning within a unified architecture, the platform supports both individual growth and organizational workforce optimization. The following examples illustrate representative, non-limiting applications of the disclosed system.

In some embodiments directed to early-career individuals, including students in colleges, universities, or training programs, an individual creates a profile through the Individual GUI Engine 106 by providing biographical information and uploading materials such as resumes, coursework, projects, certifications, portfolios, or other digital artifacts. The Skills Inference Engine 112 extracts Generic Skills 206 and infers Power Skills 208 from these materials using automated feature extraction and natural language processing techniques. The Skills Assessment Engine 126 administers Assessment Items calibrated for entry-level or future career paths, generating structured signals that update proficiency estimates and associated confidence levels. A verified JobSkillsPrint 620 is produced and stored as a structured representation of the individual's skills. The Job Success Fit Engine 128 computes JobSuccessFit scores 1108 between the individual's JobSkillsPrint 620 and a plurality of available internships, apprenticeships, or entry-level job positions, and presents ranked opportunities. The Career Pathway Engine 130 generates an Action Plan comprising targeted exercises, recommended coursework, certifications, or micro-practice activities to address identified skill gaps. As additional evidence is collected, the individual's proficiency estimates and JobSuccessFit scores 1108 are automatically recalculated, thereby empowering student growth and improving readiness for workforce entry.

In some embodiments directed to improving placement and matching accuracy, individuals and job positions are each represented as standardized skill vectors or structured verified JobSkillsPrints 620. Employers create job position profiles through the Employer GUI Engine 116, and required skills are inferred and normalized by the Skills Ontology Engine 114. The Job Success Fit Engine 128 computes compatibility metrics between individual skill vectors and job requirement vectors using weighted similarity calculations or trained predictive models. Individuals are ranked according to their computed JobSuccessFit scores 1108, and employers may filter or prioritize individuals exceeding predefined thresholds or demonstrating strengths in critical competencies. Because both individual and job profiles are continuously updated through new assessments and training data, the system dynamically refines matching outcomes, resulting in stronger alignment between individual capabilities and job expectations compared to static resume or keyword-based processes.

In some embodiments directed to career advancement, internal mobility and talent development, an employee maintains a verified JobSkillsPrint 620 reflecting verified proficiency estimates across both Generic Skills and Power Skills. For a desired future role, such as a supervisory or managerial position, the system compares the employee's current proficiency distribution to the role's required skill thresholds. The Career Pathway Engine 130 identifies gaps and generates a personalized development plan that may include additional assessments, structured learning content, or behaviorally oriented micro-exercises. As the employee completes recommended actions or provides new evidence, the Skills Assessment Engine 126 updates the employee's proficiency distributions and the Job Success Fit Engine 128 recalculates readiness metrics. When confidence levels or proficiency thresholds are satisfied, the system may indicate that the employee is prepared for advancement, thereby supporting data-driven promotion and mobility decisions.

In some embodiments directed to leadership succession and executive readiness, the system evaluates individuals for director-level, senior leadership, or C-suite responsibilities, including management of business units, entire organizations, or portfolios of companies. Assessment Items may simulate strategic and organizational scenarios such as cross-functional coordination, capital allocation decisions, stakeholder communication, enterprise risk management, or management of inter-company relationships. These items generate signals associated with higher-order competencies including Leadership, Strategic Communication, Systems Thinking, Analytical Thinking, and AI Fluency. Proficiency estimates are updated using probabilistic inference models, and JobSuccessFit 1108 calculations incorporate metadata describing organizational attributes such as company size, industry, operational complexity, field of operation, and relationships with other entities. The system may produce readiness indicators for specific executive roles or company types and generate targeted development plans, such as simulations, mentorship recommendations, or strategic exercises, thereby supporting structured succession planning and evidence-based identification of leadership individuals, including those responsible for overseeing a portfolio of companies.

In some embodiments directed to enterprise-level workforce planning, the system aggregates verified JobSkillsPrints 620 across a population of employees to generate an organization-wide skills map. Statistical analyses identify distributions of proficiencies, coverage gaps for strategic initiatives, and employees whose skills align with current or anticipated roles. The Job Success Fit Engine 128 evaluates each employee's compatibility with open or projected positions and identifies individuals whose scores exceed or approach defined thresholds. For near-threshold employees, the Career Pathway Engine 130 generates targeted development actions intended to close specific gaps and to develop talent for current and future needs. Administrative interfaces may present readiness indicators, succession pipelines, and skill trend analytics to managers, enabling proactive staffing decisions, reducing external hiring costs, and improving retention through continuous development.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device.

Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2, the method 300 of FIG. 3, the method 400 of FIG. 4, the method 500 of FIG. 5, the method 600 of FIG. 6, the method 700 of FIG. 7, the method 800 of FIG. 8, the method 900 of FIG. 9, the method 1100 of FIG. 11, and the method 1300 of FIG. 13.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the Automated Inference and Assessment System and the Individual Devices 102 and/or the Employer Devices 104 of FIG. 1.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some embodiments, a cloud computing environment, such as Microsoft Azure™ Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some embodiments, the cloud storage database, such as the Google™ Cloud Storage or Amazon™ Elastic File System (EFS™), may store processed and unprocessed data supplied by systems described herein. For example, the contents of the Feature Vector Store 134 of FIG. 1, and/or the training data and resulting weighted feature vectors of the fine-tuned Trained ML Models 105 of FIG. 1 may be maintained in a database structure.

The systems described herein may communicate with the cloud computing environment through a secure gateway. In some embodiments, the secure gateway includes a database querying interface, such as the Google BigQuery™ platform or Amazon RDS™. The data querying interface, for example, may support access by the Automated Inference and Assessment System to at least portions of the data of the Feature Vector Store 134 of FIG. 1.

In some embodiments, this disclosure provides computer-implemented methods for assessing an individual's skills by training a plurality of AI models to assess a set of skills for an individual, wherein the training of each respective AI model of the plurality of AI models comprises providing information about a plurality of individuals, information about a plurality of skills, and information about the skills of the plurality of individuals; obtaining, via a user interface, information about an individual of the plurality of individuals; extracting, via a first AI model of the plurality of AI models, a first set of skills of the plurality of skills from the information about the individual; inferring, via a second AI model of the plurality of AI models, a second set of skills of the plurality of skills derived from the first set of skills; combining the first set of skills of the plurality of skills and the second set of skills of the plurality of skills into a third set of skills; and assessing, via a third AI model of the plurality of AI models, the individual's proficiency of at least one skill of the third set of skills, wherein, the individual's proficiency in at least one skill of the third set of skills is assigned a confidence level, through the user interface, the assessing prompts the individual in real time with an assessment item of a plurality of assessment items to provide additional information related to the at least one skill of the third set of skills; the individual, through the user interface, provides a response to the assessment item of the plurality of assessment items, and the confidence level of the individual's proficiency in the at least one skill of the third set of skills is adjusted.

In some embodiments, the assessment item of the plurality of assessment items is a prototype question.

In some embodiments, the method further comprises the step of generating, via a fourth AI model of the plurality of AI models, a new assessment item tailored to the individual based at least partially on the individual's proficiency in the at least one skill of the third set of skills.

In some embodiments, the assessment item of the plurality of assessment items simulates a realistic workplace scenario.

In some embodiments, the method further comprises the step of, via a fifth AI model of the plurality of AI models, updating the individual's proficiency in the at least one skill of the third set of skills based on the response to the assessment item of the plurality of assessment items.

In some embodiments, the individual's proficiency in the at least one skill of the third set of skills is a probability distribution.

In some embodiments, the individual's proficiency in the at least one skill of the third skills is at least partially calculated based on a performance level descriptor.

In some embodiments, the method further comprises the step of identifying, via a sixth AI model of the plurality of AI models, at least one skill from the third set of skills for the individual to improve their proficiency.

In some embodiments, the at least one skill from the third set of skills is identified as a skill required for the individual's career advancement.

In some embodiments, the method further comprises the step of providing, via the user interface, an action item designed to improve the individual's proficiency in the at least one skill of the third set of skills.

In some embodiments, the action item is an activity that can be performed during the individual's regular work routine.

In some embodiments, the method further comprises the step of continuously training the third AI model of the plurality of AI models with the additional information related to the at least one skill of the third set of skills provided by the individual.

In some embodiments, the skills of the information about the plurality of skills is a skills ontology, organizing relationships between the plurality of skills.

In some embodiments, the relationships between the plurality of skills are comprised of at least one prerequisite skill of at least one skill of the plurality of skills.

In some embodiments, the skills ontology is comprised of performance level indicator for at least one skill of the plurality of skills.

In some embodiments, the second AI model of the plurality of AI models infers the second set of skills from the first set of skills based on skill-to-skill inferences within the skills ontology.

In some embodiments, the at least one skill of the third set of skills is a soft skill.

In some embodiments, the method further comprises the step of continuously training the plurality of AI models with information provided by at least one individual of the plurality of individuals.

In some embodiments, the information about the individual is comprised of a resume.

In some embodiments, the obtaining, via a user interface, information about the individual of the plurality of individuals is comprised of entering information about the individual in a free-form text.

In some embodiments, the method further comprises the steps of obtaining, via a user interface, information about a job position; extracting, via a seventh AI model of the plurality of AI models, a seventh set of skills of the plurality of skills from the information about the job position, the seventh set of skills applicable to performance in the job position; inferring, via an eighth AI model of the plurality of AI models, an eighth set of skills of the plurality of skills derived from the seventh set of skills, wherein the eighth set of skills is related to the seventh set of skills through a standardized skills ontology; combining the seventh set of skills and the eighth set of skills into a ninth set of skills, wherein the at least one skill of the third set of skills is also present in the ninth set of skills; and assessing through a ninth AI model of the plurality of AI models, the individual's fitness for the job position based at least partially on the individual's proficiency level in the at least one skill of the third set of skills and the proficiency level required in the by the job.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1.-21. (canceled)

22. A computer-implemented method for operating an artificial intelligence skill assessment platform, the method comprising:
maintaining, by one or more processors, a standardized skills ontology that stores relationships among a plurality of skills, the relationships including at least skill-to-skill relationships for inferring skills from other skills;
receiving, through a user interface, profile information for an individual;
extracting, by a first artificial intelligence model, a first set of skill records from the profile information, each skill record of the first set of skill records identifying a respective skill and evidence from the profile information supporting the respective skill;
generating, by a second artificial intelligence model using the standardized skills ontology, a second set of skill records inferred from the first set of skill records based on the skill-to-skill relationships stored in the standardized skills ontology;
generating, by the one or more processors, an individual skill profile data structure by combining the first set of skill records and the second set of skill records;
for at least one skill represented in the individual skill profile data structure, assigning, by a third artificial intelligence model, a proficiency value and a confidence value based at least in part on the evidence associated with the at least one skill;
determining, by the one or more processors, that the confidence value for the at least one skill satisfies a criterion for additional assessment;
selecting, by an assessment engine, an assessment item associated with the at least one skill;
transmitting, through the user interface, the assessment item to the individual;
receiving, through the user interface, a response to the assessment item; and
updating, by the one or more processors, the individual skill profile data structure by adjusting at least one of the proficiency value or the confidence value for the at least one skill based on the response.

23. The method of claim 22, wherein the individual skill profile data structure comprises a job skill for the individual.

24. The method of claim 22, wherein the assessment item comprises a prototype question selected from an item bank associated with the at least one skill.

25. The method of claim 22, wherein selecting the assessment item comprises selecting a question that simulates a workplace scenario associated with the at least one skill.

26. The method of claim 22, further comprising generating, by a fourth artificial intelligence model, a new assessment item tailored to the individual based at least in part on the proficiency value or the confidence value for the at least one skill.

27. The method of claim 22, wherein updating the individual skill profile data structure comprises updating a probability distribution representing the individual's proficiency in the at least one skill.

28. The method of claim 22, wherein assigning the proficiency value comprises mapping the at least one skill to a performance level descriptor stored in association with the standardized skills ontology.

29. The method of claim 22, wherein the standardized skills ontology stores prerequisite relationships among skills, and wherein generating the second set of skill records comprises inferring at least one skill based on a prerequisite relationship with a skill in the first set of skill records.

30. The method of claim 22, wherein the standardized skills ontology stores cross-functional relationships among skills, and wherein generating the second set of skill records comprises inferring at least one cross-functional skill from at least one skill in the first set of skill records.

31. The method of claim 22, wherein at least one skill represented in the individual skill profile data structure comprises a soft skill.

32. The method of claim 22, further comprising identifying, by a recommendation engine, at least one skill represented in the individual skill profile data structure for improvement by the individual based at least in part on the proficiency value for the at least one skill.

33. The method of claim 32, further comprising transmitting, through the user interface, an action item configured to improve the individual's proficiency in the at least one skill.

34. The method of claim 33, wherein the action item comprises an activity for completion during a regular work routine of the individual.

35. The method of claim 22, further comprising:
receiving, through an employer user interface, job position information for a job position;
extracting, by a job-position artificial intelligence model, a set of required skill records from the job position information;
generating, using the standardized skills ontology, an inferred set of required skill records associated with the job position;
generating a job skill profile data structure by combining the set of required skill records and the inferred set of required skill records; and
calculating, by a job-fit engine, a fit value for the individual relative to the job position based at least in part on a comparison between the individual skill profile data structure and the job skill profile data structure.

36. The method of claim 35, wherein calculating the fit value comprises comparing the proficiency value for the at least one skill of the individual with a proficiency level required for a corresponding skill in the job skill profile data structure.

37. A computer-implemented method for generating an artificial intelligence-based skill profile for an individual, the method comprising:
receiving, by one or more processors through a user interface, information associated with an individual;
identifying, by a first artificial intelligence instance, a first set of skills associated with the individual based at least in part on the information associated with the individual;
identifying, by a second artificial intelligence instance, a second set of skills associated with the individual based at least in part on the first set of skills and relationship information among a plurality of skills;
generating, by the one or more processors, a skill profile for the individual based at least in part on the first set of skills and the second set of skills; and
determining, by a third artificial intelligence instance, a proficiency associated with at least one skill included in the skill profile.

38. The method of claim 37, further comprising
maintaining, by one or more processors, a standardized skills ontology that stores relationships among a plurality of skills, the relationships including at least skill-to-skill relationships for inferring skills from other skills,
wherein the standardized skills ontology comprises a hierarchy of skills and subskills and weighted relationships between skills or subskills, each weighted relationship indicating an influence that an assessment of one skill or subskill has on an assessment of another skill or subskill; and
receiving, through a user interface, profile information for an individual,
wherein the relationship information among the plurality of skills is stored in a skills ontology, and wherein the standardized skills ontology comprises a hierarchy of skills and subskills and weighted relationships between skills or subskills, each weighted relationship indicating an influence that an assessment of one skill or subskill has on an assessment of another skill or subskill.

39. The method of claim 38, wherein the skills ontology includes at least one relationship selected from the group consisting of a prerequisite relationship, a related-skill relationship, a skill-category relationship, and a performance-level relationship.

40. The method of claim 37, wherein the information associated with the individual comprises at least one of resume information, work experience information, education information, certification information, project information, free-form text, or profile information entered through the user interface.

41. The method of claim 37, wherein identifying the first set of skills comprises extracting one or more expressly stated skills from the information associated with the individual.

42. The method of claim 37, wherein identifying the second set of skills comprises inferring one or more skills not expressly stated in the information associated with the individual.

43. The method of claim 37, wherein the proficiency associated with the at least one skill comprises a proficiency level, a score, a classification, a ranking, a probability, or a probability distribution.

44. The method of claim 37, further comprising assigning a confidence level to the proficiency associated with the at least one skill.

45. The method of claim 37, further comprising identifying at least one skill for improvement based at least in part on the skill profile.

46. The method of claim 45, further comprising generating an action item associated with improving the at least one skill for improvement.

47. The method of claim 46, wherein the action item comprises an activity configured for completion during a work routine of the individual.

48. The method of claim 37, wherein at least one skill included in the skill profile comprises a soft skill.
